(19) United States
(12) Patent Application Publication
(10) Pub. No.: US 2025/0152180 A1
(43) Pub. Date: May 15, 2025

(54) DELIVERY CATHETERS WITH ANTI-REFLUX OCCLUDER AND ANTI-STRETCH FEATURES

(71) Applicant: Neuravi Limited, Galway (IE)

(72) Inventors: David VALE, Barna (IE); Alan CARNEY, Galway (IE)

(73) Assignee: Neuravi Limited, Galway (IE)

(21) Appl. No.: 18/910,218

(22) Filed: Oct. 9, 2024

Related U.S. Application Data
(60) Provisional application No. 63/548,004, filed on Nov. 10, 2023.
(60) Provisional application No. 63/548,006, filed on Nov. 10, 2023.

(51) Int. Cl. A61B 17/12; A61B 17/00; A61B 90/00

(52) U.S. Cl. CPC A61B 17/12186; A61B 17/00234; A61B 17/12109; A61B 90/39; A61B 2017/00238; A61B 2017/00305; A61B 2017/00398; A61B 2017/1205; A61B 2090/3966

(57) ABSTRACT

Delivery catheters for occluding aneurysms, the delivery catheter including a catheter tubing extending a length. In some configurations the catheter may include a reflux-occluder section having a third diameter greater than the first diameter and the second diameter. The reflux-occluder section is disposed along the length of the catheter tubing between the proximal portion and the distal portion. Certain implementations of the catheters may include an expansile section having a collapsed configuration with a first diameter and an expanded configuration with a second diameter, the second diameter sized to prevent reflux of glue proximal to the expansile section within a vessel. Configuration of the catheter may include a cyclic pump to create a fluid column proximate the distal end and positioned between the distal end and the glue to prevent reflux of the glue proximal to the distal end.

### CROSS REFERENCE TO RELATED APPLICATION

The present application claims benefit of priority to U.S. Provisional Patent Application No. 63/548,004 filed Nov. 10, 2023, and U.S. Provisional Patent Application No. 63/548,006 filed Nov. 10, 2023. The entire contents of which each are hereby incorporated by reference.

### FIELD OF INVENTION

The present disclosure generally relates to devices and methods for delivering an embolic glue or other fluid embolic agent to a treatment site. More specifically, the present disclosure relates to catheters with anti-reflux occlusion sections, catheters with anti-stretch features and/or catheters with cyclic pulsing to prevent reflux.

### BACKGROUND

Certain vascular procedures require the embolization of vessels to prevent blood flow to that particular area of the body. In neurovascular procedures, this embolization can include placing an embolic device or material in a target vessel to occlude blood flow, for example, to an aneurysm, to an arteriovenous malformation (AVM), to a chronic subdural hematoma (CSDH), or other malformation. Precise placement embolic device or material is critical to prevent inadvertent migration, which can cause significant problems including a stroke if a non-target vessel is occluded.

One method of embolizing a target vessel is to provide a fluid embolic agent like a cyanoacrylate glue (e.g., n-Butyl cyanoacrylate (NBCA)) to the target vessel. These types of glues polymerize when in contact with blood, thus occluding flow and creating a flexible embolism. As stated above, ensuring delivery of this glue to the precise target site, and ensuring that the glue stays in the precise target site, is critical to the operation. Issues can occur when the cyanoacrylate glue refluxes proximal of the delivery catheter. This can cause glue to migrate to untargeted areas of the vasculature, and can also make it difficult to remove the delivery catheter. Even if the glue does not reflux past the delivery catheter, the risk remains that the glue polymerizes and causes the distal tip of the catheter to become stuck within the glue, making it more difficult to retrieve the catheter after embolization. These and other problems exist.

### SUMMARY

It is an object of the present designs to provide devices and methods to overcome the above-stated issues. One aspect of the present disclosure provides a delivery catheter. A proximal portion of the catheter tubing can have a first diameter and a distal portion of the catheter tubing can have a second diameter. The delivery catheter can include a reflux-occluder section having a third diameter greater than the first diameter and the second diameter. The reflux-occluder section can be disposed along the length of the catheter tubing between the proximal portion and the distal portion.

The first diameter of the catheter tubing can be larger than the second diameter of the catheter tubing. The reflux-occluder section can be a bulge in the catheter tubing. The reflux-occluder section can be compressible for insertion into a vessel and expandable to the third diameter. The catheter tubing can include a braid extending along the length of the catheter tubing. A distal end of the braid can be positioned within the proximal portion of the catheter tubing. A distal end of the braid can be positioned within the reflux-occluder section. A distal end of the braid can be positioned within the distal portion of the catheter tubing. A distal end of the braid can include a braid attachment. The braid attachment can be a ring having an atraumatic distal end. The braid attachment can include a radiopaque material.

The reflux-occluder section can be one or more baffles extending from the catheter tubing. The one or more baffles can include a spiraling baffle that spirals around a longitudinal axis of the delivery catheter. The one or more baffles can include a first spiraling baffle and a second spiraling baffle offset radially from the first spiraling baffle. The one or more baffles can include a ring baffle extending radially from the catheter tubing. The one or more baffles can include a first ring baffle having a first baffle diameter and a second ring baffle having a second baffle diameter. The first baffle diameter can be smaller than the second baffle diameter.

The catheter tubing can include a braid extending along the length of the catheter tubing. The third diameter can be less than a diameter of a target a vessel.

One aspect of the present disclosure provides a delivery catheter. The delivery catheter can include a catheter tubing extending a length and comprising a braid extending along the length of the catheter tubing. The delivery catheter can include a reflux-occluder section disposed along the length of the catheter tubing between a proximal portion and a distal portion of the catheter tubing, wherein a distal end of the braid is positioned within the reflux-occluder section. The catheter tubing can include an axial fiber.

One aspect of the present disclosure provides a method. The method can include positioning a reflux-occluder section proximal to the targeted site such that the reflux-occluder section extends radially from a catheter tubing of the delivery catheter and contacts a vessel wall proximate the targeted site. The method can include delivering a glue through the catheter tubing. The method can include limiting, via the reflux-occluder section, the glue from refluxing proximal to the reflux-occluder section. The method can include withdrawing the delivery catheter from the main vessel. The reflux-occluder section can include an occluder diameter greater than a proximal diameter of a proximal portion of the catheter tubing, and the occluder diameter can be greater than a third diameter of a distal portion of the catheter tubing.

Another aspect of the present disclosure provides a delivery catheter. The delivery catheter includes a length of catheter tubing. The delivery catheter includes a non-expansile section positioned along the length. The delivery catheter includes an expansile section disposed along the length distal to the non-expansile section. The delivery catheter includes a restrictor disposed within a lumen of the catheter tubing distal to the expansile section. The expansile section has a collapsed configuration with a first diameter and an expanded configuration with a second diameter. The second diameter is sized to limit reflux of a fluid embolic agent proximal to the expansile section within a vessel. The catheter tubing, the non-expansile section, and the expansile section share a single lumen.

Yet another aspect of the present disclosure provides a delivery catheter. The delivery catheter includes a length of catheter tubing. The delivery catheter includes an expansile section positioned along the length. The expansile section includes a fixed proximal collar and a membrane having an open end. The expansile section has a collapsed configuration with a first diameter and an expanded configuration with a second diameter. The second diameter is sized to limit reflux of fluid embolic agent within a vessel proximal to the expansile section.

Still another aspect of the present disclosure provides a delivery catheter. The delivery catheter includes catheter tubing having a distal end. The delivery catheter includes agent tubing in fluid communication with the distal end of the catheter tubing. The delivery catheter includes an agent pump to drive fluid embolic agent through the agent tubing. The delivery catheter includes fluid tubing in fluid communication with the distal end of the catheter tubing. The delivery catheter includes a cyclic pump to drive a second fluid to the distal end of the catheter tubing in cyclic pulses. The cyclic pulses create a fluid column proximate the distal end positioned between the distal end and the fluid embolic agent to limit reflux of the fluid embolic agent proximal to the distal end. The second fluid is a different material than the fluid embolic agent.

Other aspects of the present disclosure will become apparent upon reviewing the following detailed description in conjunction with the accompanying figures. Additional features or manufacturing and use steps can be included as would be appreciated and understood by a person of ordinary skill in the art.

### BRIEF DESCRIPTION OF THE DRAWINGS

The above and further aspects of this disclosure are further discussed with reference to the following description in conjunction with the accompanying drawings, in which like numerals indicate like structural elements and features in various figures. The drawings are not necessarily to scale, emphasis instead being placed upon illustrating principles of the disclosure. The figures depict one or more implementations of the inventive devices, by way of example only, not by way of limitation. It is expected that those of skill in the art can conceive of and combine elements from multiple figures to better suit the needs of the user.

FIG. 1 is a diagram of a delivery catheter being advanced through the vasculature, according to aspects of the present disclosure;

FIG. 2A is a side view of a delivery catheter having a reflux-occluder section and a braid extending proximal to the reflux-occluder section, according to aspects of the present disclosure;

FIG. 2B is a side view of a delivery catheter having a reflux-occluder section and a braid extending to the reflux-occluder section, according to aspects of the present disclosure;

FIG. 2C is a side view of a delivery catheter having a reflux-occluder section and a braid extending distal to the reflux-occluder section, according to aspects of the present disclosure;

FIG. 3A is a perspective view of a delivery catheter having a reflux-occluder section with a spiraling baffle, according to aspects of the present disclosure;

FIG. 3B is a perspective view of a delivery catheter having a reflux-occluder section with ring baffles, according to aspects of the present disclosure;

FIG. 3C is a cross-sectional view of the reflux-occluder section of the delivery catheter in FIG. 3B, according to aspects of the present disclosure;

FIG. 4 is a perspective view of a delivery catheter having proximal braid and an axial fiber, according to aspects of the present disclosure;

FIG. 5 is a flowchart showing an example method of using a delivery catheter, according to aspects of the present disclosure;

FIG. 6 is a diagram of another example delivery catheter being advanced through the vasculature, according to aspects of the present disclosure;

FIG. 7A is a side cross-sectional view of the example delivery catheter of FIG. 6 having an expansile section, according to aspects of the present disclosure;

FIG. 7B is an end view of the delivery catheter of FIG. 7A, according to aspects of the present disclosure;

FIG. 7C is a side view of the delivery catheter of FIG. 7A with the expansile section expanded, according to aspects of the present disclosure;

FIG. 8A is a side view of a delivery catheter with an umbrella-style expansile section, according to aspects of the present disclosure;

FIG. 8B is a side view of the delivery catheter of FIG. 8A with the expansile section expanded, according to aspects of the present disclosure;

FIG. 8C is a radial cross-sectional view of the delivery catheter of FIG. 8B along lines 8B-8B, according to aspects of the present disclosure;

FIG. 8D is a side view of another delivery catheter with an umbrella-style expansile section with a frame having an open end, the expansile section being in an expanded configuration, according to aspects of the present disclosure;

FIG. 8E is a radial cross-sectional view of the delivery catheter of FIG. 8D along lines 8E-8E, according to aspects of the present disclosure;

FIG. 8F is a side view of the delivery catheter of FIG. 8D with the expansile section in a collapsed configuration, according to aspects of the present disclosure;

FIG. 8G is an example frame for an umbrella-style expansile section, according to aspects of the present disclosure; and

FIGS. 9A-4E are side views of a catheter that utilizes a fluid column to prevent reflux of fluid embolic agent, according to aspects of the present disclosure. FIG. 9A is a side view of the catheter; FIG. 9B is a side view of the catheter ejecting fluid embolic agent; FIG. 9C is a side view of the catheter expelling a fluid column; FIG. 9D is a side view of the catheter with the fluid column discontinued; and FIG. 9E is a side view of a catheter with tubing apertures to allow fluid to exit the catheter proximal to the distal end of the catheter.

### DETAILED DESCRIPTION

Specific examples of the present disclosure are now described in detail with reference to the Figures, where identical reference numbers indicate elements which are functionally similar or identical. The examples address many of the deficiencies associated with traditional clot retrieval aspiration catheters, such as poor or inaccurate deployment to a target site and ineffective clot removal.

The designs herein are for catheters used in embolic procedures that use glues, such as n-Butyl cyanoacrylate (NBCA) or other cyanoacrylate glues, or other liquid embolic agents such as copolymers like Onyx, Squid and PHIL, or other particles to occlude a vessel. The designs herein provide several advantages over prior catheter designs. For example, a typical rubber or non-rubber catheter may be nothing more than a cylindrical tube extending along a length. Such a catheter can be inserted to a treatment site to deliver the embolic glue, but issues exist. For one, the existing catheters have poor pushability and are prone to stretching and breaking if the tip gets stuck in the polymerized glue. Further, since the prior catheter systems were mere cylindrical tubes, nothing in the design prevented reflux of the glue up the length of catheter. The designs described herein provide improvements to the catheter system to alleviate the issues with pushability, retractability, and/or reflux.

Accessing the various vessels within the vascular, whether they are coronary, pulmonary, or cerebral, involves well-known procedural steps and the use of a number of conventional, commercially-available accessory products. These products, such as angiographic materials, mechanical thrombectomy devices, microcatheters, and guidewires are widely used in laboratory and medical procedures. When these products are employed in conjunction with the devices in the description below, their function and exact constitution are not described in detail. While the description is in many cases in the context of thrombectomy treatments in intercranial arteries, the disclosure may be adapted for other procedures and in other body passageways as well.

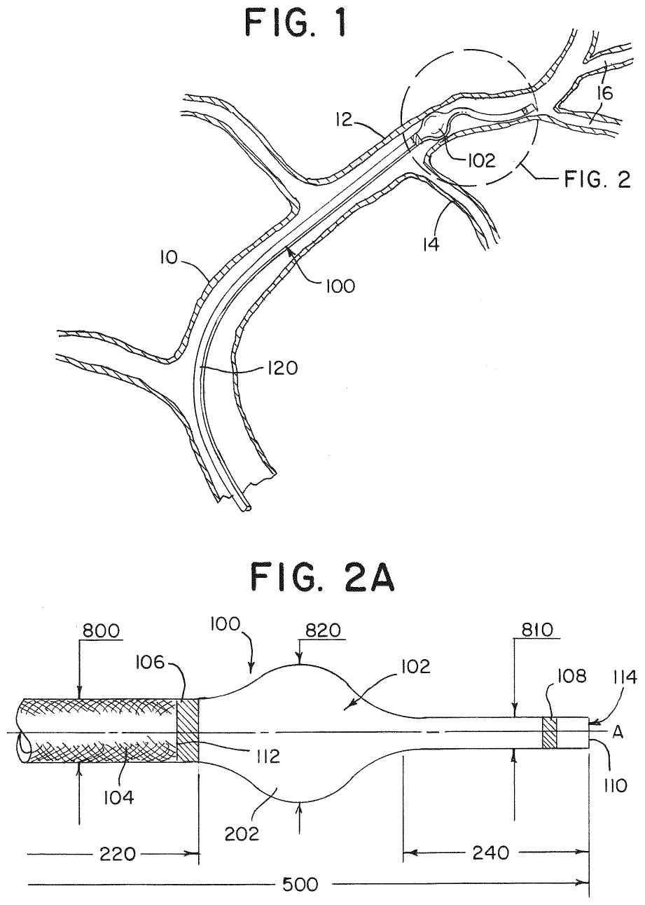

Turning to the figures, FIG. 1 illustrates a possible sequence for using a delivery catheter 100 to approach a targeted site 16 through a main vessel 10. In the example shown, the targeted site 16 is one or more vessel branches distal to a non-targeted site 14, meaning that occlusion at the location of the distal end of the delivery catheter 100 would prevent blood flow proximal to that site. The targeted site 16 in this example shows another vessel branch that would, therefore, not be affected by occlusion distal to that site. The delivery catheter 100 can include a highly flexible catheter tubing 120 such that it is capable of navigating the M1 or other tortuous regions of the neurovascular system to reach proximate the targeted site 16. As will be described in greater detail herein, the delivery catheter 100 can include a reflux-occluder section 102 that has a diameter that assists to limit or prevent reflux of a glue that may be delivered via the catheter. Any reference to a “glue” in the instant disclosure is not limiting, as the delivery catheters described herein can prevent many types of fluid embolic agents from refluxing. Fluid embolic agents can, therefore, include glues such as n-Butyl cyanoacrylate (NBCA) or other cyanoacrylate glues, other agents such as copolymers like Onyx, Squid and PHIL, or other particles to occlude a vessel. Accordingly, when the term “glue” is used herein, it will be understood that the other fluid embolic agents can be substituted for “glue.” In some examples, this reflux-occluder section 102 can contact a vessel wall 12 so as to limit reflux of glue proximate the reflux-occluder section 102. It is also contemplated that the reflux-occluder section 102 has a diameter slightly less than the diameter of the vessels in the area, as full peripheral contact may not be necessary to limit reflux of the glue proximal to the reflux-occluder section 102 since the glue may be more viscous that other fluids at the target site.

FIGS. 2A-2C depict a delivery catheter 100 having a reflux-occluder section 102 that is a bulge 202 in the catheter tubing 120. For description purposes, the delivery catheters 100 can be considered to have a length 500 with a proximal portion 220 that is proximal to the reflux-occluder section 102, and a distal portion 240 that is distal to the reflux-occluder section 102. The delivery catheters 100 shown in FIGS. 2A-2C show an example wherein the proximal portion 220 has a proximal diameter 800 that is greater than a distal diameter 810 of the distal portion 240; wherein the reflux-occluder section 102 has a diameter 820 that is greater than both the proximal diameter 800 and distal diameter 810 (i.e., the bulge 202). By having a proximal portion 220 with a larger diameter tubing than the distal portion 240, the distal portion 240 can act as a bottleneck for the glue flowing through a lumen 114 of the delivery catheter 100. This bottleneck can create a positive pressure within the reflux-occluder section 102 as glue is pushed through the catheter, allowing the reflux-occluder section 102 to expand to contact the vessel wall 12 (see FIG. 1). In other examples, the proximal diameter 800 and distal diameter 810 can be the same diameter.

Another feature of the delivery catheters 100 shown in FIGS. 2A-2C is that the reflux-occluder section 102 (i.e., bulge 202) can be formed with only a single lumen 114 that extends along the length 500 of the catheter through each of the proximal portion 220, reflux-occluder section 102, and distal portion 240. This is contrary to a traditional balloon catheter, since balloon catheters have multiple lumens, one to inflate the balloon and another to deliver instruments distal to the balloon. In the examples shown in FIGS. 2A-2C, there is no need for a separate lumen as the lumen 114 is used to deliver the glue to a distal tip 110 of the delivery catheter 100. In some examples, the bulge 202 can be made to have a low radial force, so that it can collapse if advanced into a vessel slightly smaller than its diameter 820. The internal pressure of the glue injection can prevent this collapsible bulge 202 from collapsing under the external pressure of glue that may be attempting to reflux proximal to the reflux-occluder section 102. This is another advantage of the systems shown in FIGS. 2A-2C over traditional balloon style catheters, in that the substance being delivered can create the positive pressure that prevents reflux of that very same substance proximally.

Referring now specifically to FIG. 2A, the figure shows an example delivery catheter 100 having a reflux-occluder section 102 with a braid 104 extending proximal to the reflux-occluder section 102. The braid 104 can provide axial strength to the catheter tubing 120 to increase its pushability through the vasculature. The braid 104 can be made of a reinforcing material such as metal wiring (e.g., stainless steel, nitinol, etc.), para-aramids, fiberglass, carbon fiber, and the like. A distal end 112 of the braid 104 can include a braid attachment 106 that can be a ring that surrounds the catheter tubing 120 to both stably attach the braid 104 to the tubing and provide an automatic profile at the end of the braid 104. In some instances, the braid attachment 106 can include a radiopaque material that can enable an operator, such as a physician, to determine via radiography where the proximal end of the reflux-occluder section 102 is positioned within the vasculature. The radiopaque material can include high-density materials such as gold or platinum.

FIG. 2B is a side view of a delivery catheter 100 having a reflux-occluder section 102 with a braid 104 extending to the reflux-occluder section 102. The braid 104 can be substantially similar to the braid described above for FIG. 2A, but the positioning of the distal end 112 of the braid is within the bulge 202 area. The braid 104 can end as shown within the bulge 202 area. The delivery catheter 100 can include a braid attachment 106, which can also act as a means to prevent the bulge 202 from expanding beyond a predetermined diameter (e.g., diameter 820 shown in FIG. 2A) under the internal pressure of glue injection. In other examples, any of the examples shown in FIGS. 2A-2C could additionally or alternatively include another expansion-limiting feature, such as a fiber built into the wall of the bulge 202. In some instances, the braid attachment 106 can include a radiopaque material that can enable an operator, such as a physician, to determine via radiography where the bulge 202 of the reflux-occluder section 102 is positioned within the vasculature.

FIG. 2C is a side view of a delivery catheter 100 having a reflux-occluder section 102 with a braid 104 extending distal to the reflux-occluder section 102. The braid 104 can be substantially similar to the braid described above for FIGS. 2A and 2B, but the positioning of the distal end 112 of the braid 104 is distal to the bulge 202. The braid material can act as a means to prevent the bulge 202 from expanding beyond a predetermined diameter (e.g., diameter 820 shown in FIG. 2A) under the internal pressure of the glue injection. In some instances, the braid attachment 106 can include a radiopaque material that can enable an operator, such as a physician, to determine via radiography where the distal end of the reflux-occluder section 102 is positioned within the vasculature. In some examples, the braid 104 can extend proximate the distal tip 110 of the reflux-occluder section 102, enabling the operator to determine via radiography where the distal end of the delivery catheter 100 is positioned within the vasculature.

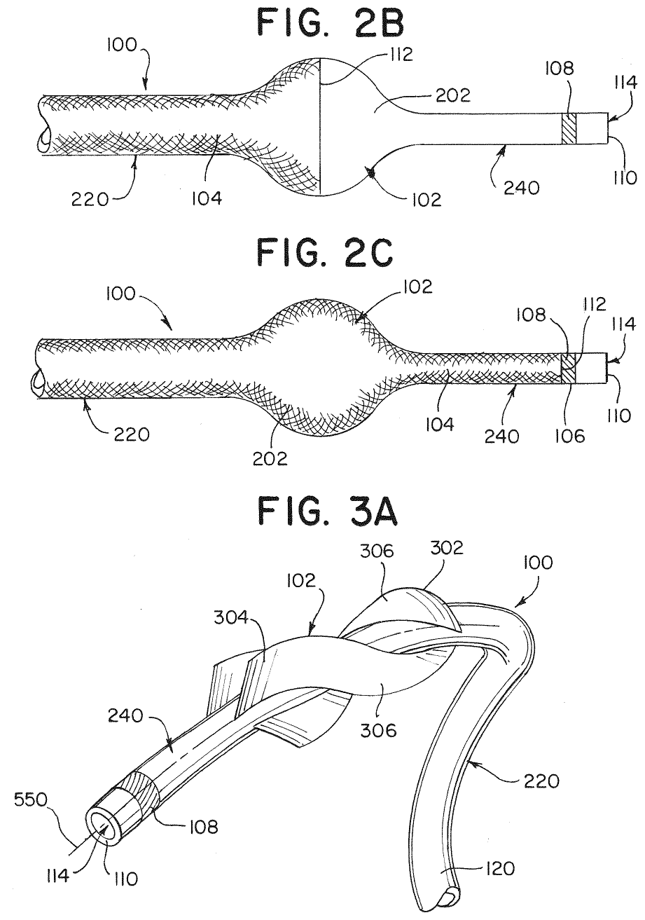

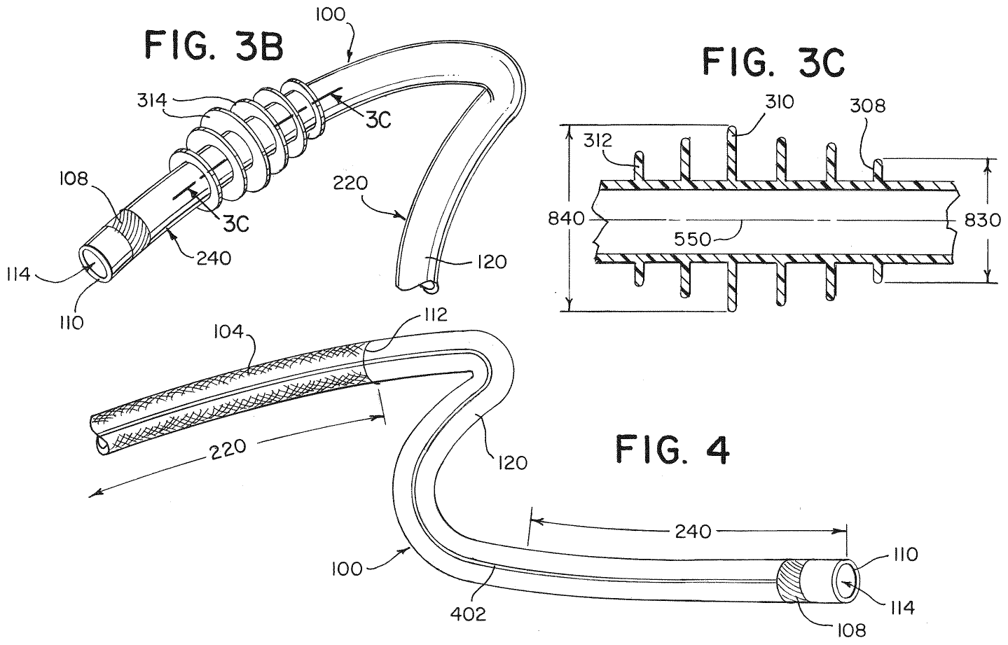

FIGS. 3A-3C depict a delivery catheter 100 having a reflux-occluder section 102 that includes one or more baffles. The delivery catheters 100 in the examples shown in FIGS. 3A-3C have a length 500 with a proximal portion 220 that is proximal to the reflux-occluder section 102 and a distal portion 240 that is distal to the reflux-occluder section 102. Like the bulge 202 described above, the baffles of the reflux-occluder section 102 can also limit or prevent glue from refluxing proximal to the reflux-occluder section 102 when the glue is administered proximal to the targeted site 16 (see FIG. 1).

Referring specifically to FIG. 3A, the reflux-occluder section 102 can include one or more spiraling baffles 306 that spiral around a longitudinal axis 550 of the delivery catheter 100. The one or more spiraling baffles 306 extend laterally from the catheter tubing 120 and proximate the vessel wall 12 (see FIG. 1). The spiraling baffles 306 not only limit or prevent reflux proximal to the reflux-occluder section 102 but also aid in centering the distal tip 110 of the catheter within the relevant vessel.

In some instances, the reflux-occluder section 102 can have a single spiraling baffle, e.g., first spiraling baffle 302 that spirals around the longitudinal axis 550 of the delivery catheter 100. In other examples, the reflux-occluder section 102 includes a second spiraling baffle 304 offset radially from the first spiraling baffle 302, as shown in FIG. 3A. First spiraling baffle 302 and second spiraling baffle 304 are collectively referred to herein as spiraling baffles 306. This example with two separate spiraling baffles 306 can create more contact areas on the vessel walls 12 (see FIG. 1). The spiraling baffles 306 can be formed from the same material as the catheter tubing 120. In other examples, the spiraling baffles 306 may be made from a more pliable, flexible material than the catheter tubing 120 so as to provide improved surface contact with the relevant vessel walls 12.

Referring now to FIGS. 3B and 3C, the reflux-occluder section 102 can include one or more ring baffles 314 extending radially from the catheter tubing 120. The ring baffles 314 work in a manner similar to the bulge 202 and the spiraling baffles 306 described above in that they can limit or prevent reflux of glue proximal to the reflux-occluder section 102. The one or more ring baffles 314 can include a first ring baffle 308 having a first baffle diameter 830 and a second ring baffle 310 having a second baffle diameter 840. The first ring baffle 308 and second ring baffle 310 are collectively referred to as ring baffles 314 herein, and for clarity the first ring baffle 308 can be considered the most distal baffle in FIG. 3C.

As shown in the cross section of FIG. 3C, the first baffle diameter 830 can be smaller than the second baffle diameter 840. In other words, the diameter of the ring baffles 314 increases as you move from distal to proximal in the reflux-occluder section 102. This variation in diameter can help to accommodate target vessels with differing diameters. Further, the smaller diameter 830 distally can allow glue to flow around the baffle in larger vessels, whereas the glue cannot flow around the larger diameter 840. As such, excess glue can become trapped between the first ring baffle 308 and the second ring baffle 310, so that it can be extracted when the delivery catheter 100 is retracted from the vessel. As shown in FIGS. 3B and 3C, the ring baffles 314 can also have a configuration where the most distal ring baffle (e.g., first ring baffle 308) has a small diameter, the centermost ring baffle (e.g., second ring baffle 310) has the largest dimeter, and then the ring baffles taper back down again such that a most proximal ring baffle (e.g., third ring baffle 312 in FIG. 3C) also has a smaller diameter. Of course, there may be more than three ring baffles. The image shown in FIGS. 3B and 3C show six ring baffles, but additional ring baffles may be incorporated.

FIG. 4 is a perspective view of a delivery catheter 100 having proximal braid 104 and an axial fiber 402 extending along the length 500 of the catheter tubing 120, although the example shown in FIG. 4 does not have a larger reflux-occluder section as shown in the prior figures, the braid 104 and axial fiber 402 shown in FIG. 4 can be applied to any one of the prior examples. As described above, the distal tip of catheters are prone to stretching and breaking if the tip gets stuck in the polymerized glue. The example delivery catheter 100 in FIG. 4 provides an improvement to the retractability of catheters by including the axial fiber 402 that extends proximate, or fully to, the distal tip 110 of the catheter tubing 120. The axial fiber 402 can material with a higher tensile strength than the catheter tubing 120, which allows the catheter tubing 120 to be flexible but also resist stretch when pulled proximally to retract from the vessel. The axial fiber 402 can comprise a liquid crystal polymer (LCP), polyethylene naphthalate (PEN), ultra-high-molecular-weight polyethylene (UHMWPE), para-aramids, fiberglass, metal wiring (e.g., stainless steel, nitinol, etc.), oriented expanded polytetrafluoroethylene (ePTFE), and the like.

In another embodiment of the catheter shown in FIG. 4, the catheter 100 comprises a liner 602 made from highly oriented polytetrafluoroethylene (PTFE), and an outer polymer jacket 604. The orientation of the polymer chains in the PTFE liner is such that the axial stiffness (or resistance to elongation) of the PTFE liner is considerably greater than that of the outer polymer jacket 604, so that an axial fiber is not required to render the catheter stretch resistant. A wrapped PTFE liner is an example of such a liner material. Specifically, the modulus of elasticity of the preferred liner 602 is more than 1,000 MPa (N/mm2), and preferably it is greater than 1,500 MPa, and most preferably close to or greater than 2,000 MPa. Because this liner 602 can be made with a very thin wall, and is on the inside of the catheter 100, its negative impact on the catheter 100 lateral flexibility can be minimized. The outer catheter jacket(s) 604 have a larger diameter and wall thickness and thus geometrically are configured to have a greater second moment of area. However, because the outer jacket 604 can be made from a polymer with a much lower modulus of elasticity than the inner liner 602, its contribution to both the axial and lateral stiffness of the catheter are lessened. Specifically, the modulus of elasticity of the preferred catheter outer jacket 604 is less than 10% of that of the liner 602, and preferably it is close to or less than 1% of that of the liner 602. This construction is hugely advantageous in that the resultant catheter is extremely flexible, yet also very stretch resistant, so that if the distal tip 110 were to become stuck in the vessel 10 post embolization, it can be safely pulled free without risk of the catheter 100 breaking and portions of it being left behind in the patient.

The liner 602 described above can be used in any of the catheter 100 designs disclosed herein to provide similar advantages to those described above. In some embodiments the liner may run to the very end of the catheter, while in others the liner 602 may stop short of the catheter tip. For example, in the catheters described in FIG. 1 and FIG. 2, the liner 602 could be stopped at or just proximal of the expanded reflux-occluder section 102.

As described throughout the disclosure, the delivery catheter 100 can include a proximal portion 220 and a distal portion 240. In some implementations, the braid 104 can be disposed on only the proximal portion 220 to increase pushability. The distal portion 240 may be free of a braid 104 to enable the distal end of the delivery catheter 100 to be soft, flexible, and atraumatic to reach the tortuous, distal regions of the neurovascular system. Further, pressure of embolic agent injection can be less at a distal end (e.g., toward the distal tip 110) of the delivery catheter 100, so that there is less need for reinforcement in this distal section. However, the braid 104 can extend all the way to the distal portion 240 as well. In some instances, the braid 104 can extend to the distal tip 110. An end of the axial fiber 402 can be secured to the catheter tubing 120 via an attachment, for example the aforementioned braid attachment 106. The braid attachment 106 can include a radiopaque material that can enable an operator, such as a physician, to determine via radiography where the distal end (e.g., distal tip 110) is positioned within the vasculature. The radiopaque material can include high-density materials such as gold or platinum. The delivery catheter 100 can additionally or alternatively include a separate radiopaque marker 108 proximate the distal tip 110 of the catheter tubing 120.

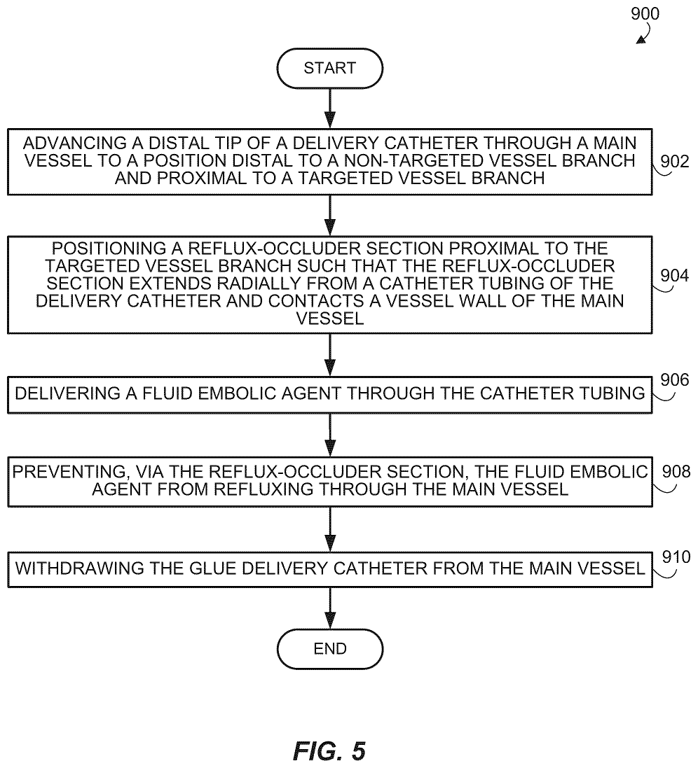

FIG. 5 is a flowchart showing an example method 900 of using a delivery catheter 100, according to aspects of the present disclosure. Method 900 includes advancing 902 a distal tip 110 of a delivery catheter 100 through a main vessel 10 to a position distal to a non-targeted site 14 and proximal to a targeted site 16. This step can be aided by a braid attachment 106 at the end of a braid 104 (for delivery catheters 100 that include a braid 104), and/or aided by a separate radiopaque marker 108 positioned proximate the distal tip 110 of the delivery catheter 100.

Method 900 includes positioning 904 a reflux-occluder section 102 proximal to the targeted site 16 such that the reflux-occluder section 102 extends radially from a catheter tubing 120 of the delivery catheter 100 and contacts at least a portion of a vessel wall 12 proximate the targeted site 16. The reflux-occluder section 102 can include an occluder diameter 820 greater than a proximal diameter 800 of a proximal portion 220 of the catheter tubing 120, and greater than a distal diameter 810 of a distal portion 240 of the catheter tubing 120.

Method 900 includes delivering 906 a fluid embolic agent through the catheter tubing 120. Method 900 includes limiting 908, via the reflux-occluder section 102, the fluid embolic agent from refluxing proximal to the reflux-occluder section 102. Method 900 includes withdrawing 910 the delivery catheter 100 from the main vessel 10. This withdrawing 910 step can also include preventing axial stretching of the catheter tubing 120 if the delivery catheter 100 includes the axial fiber 402.

The specific examples next to be described address many of the deficiencies associated with traditional clot retrieval aspiration catheters, such as poor or inaccurate deployment to a target site and ineffective clot removal.

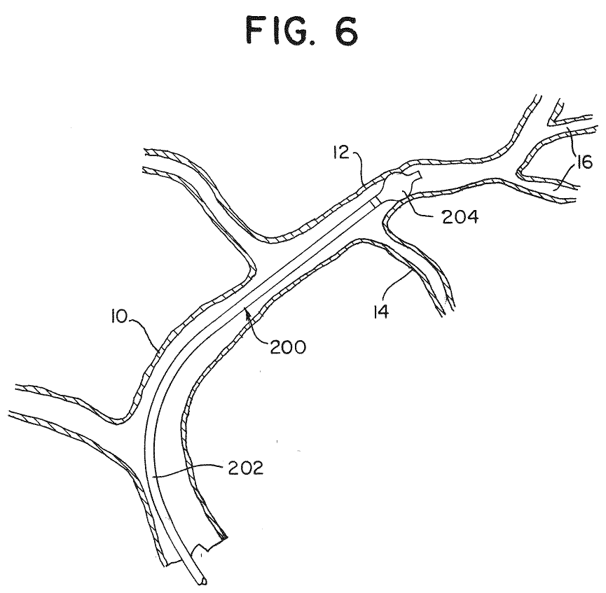

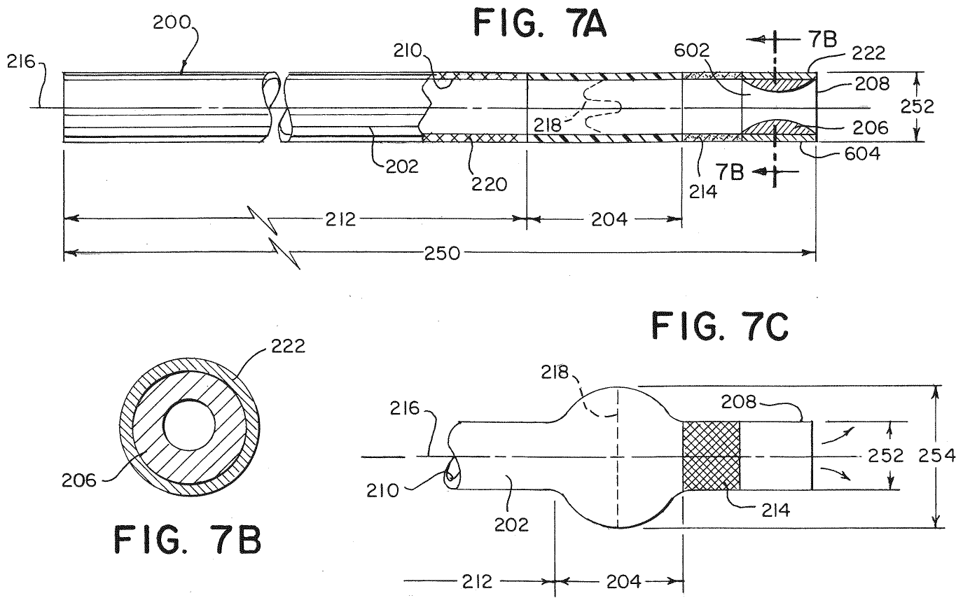

FIG. 6 illustrates a possible sequence using another example delivery catheter 200 to approach a targeted site 16 through a main vessel 10. In the example shown, the targeted site 16 is one or more vessel branches distal to a non-targeted site 14, meaning that occlusion at the location of the distal end of the delivery catheter 200 would prevent blood flow proximal to that site. The targeted site 16 in this example shows another vessel branch that would, therefore, not be affected by occlusion distal to that site. The delivery catheter 200 can include a highly flexible catheter tubing 202 such that it is capable of navigating the M1 or other tortuous regions of the neurovascular system to reach proximate the targeted site 16. As will be described in greater detail herein (see for example FIG. 7A), the delivery catheter 200 can include an expansile section 204, 304 disposed along a length 250 of the catheter 200. In some examples, this expansile section 204, 304 can contact a vessel wall 12 so as to prevent or limit reflux of glue proximate the expansile section 204, 304. Any reference to a “glue” in the instant disclosure is not limiting, as the delivery catheters described herein can prevent many types of fluid embolic agents from refluxing. Fluid embolic agents can, therefore, include glues such as n-Butyl cyanoacrylate (NBCA) or other cyanoacrylate glues, other agents such as copolymers like Onyx, Squid and PHIL, or other particles to occlude a vessel. Accordingly, when the term “glue” is used herein, it will be understood that the other fluid embolic agents can be substituted for “glue.”

FIGS. 7A-7C show a delivery catheter 200 having an expansile section 204, according to aspects of the present disclosure. The delivery catheter 200 in FIGS. 7A-7C has a length 250 of catheter tubing 202, and the catheter tubing 202 has a non-expansile section 212 and an expansile section 204 disposed along the length 250 distal to the non-expansile section. The expansile section 204 has a collapsed configuration with a first diameter 252 (see FIGS. 7A) and an expanded configuration with a second diameter 254 (see FIG. 7C). The second diameter 254 is sized to engage vessel walls 12 and prevent or limit reflux of glue proximal to the expansile section 204 within the vessel 10. The catheter tubing 202, the non-expansile section 212, and the expansile section 204 each share a single lumen 210.

The expansile section 204 comprises an elastomeric material that allows it to stretch outwardly (e.g., radially from a longitudinal axis 216) when under positive pressure. To create the positive pressure, the delivery catheter 200 can include a restrictor 206 positioned proximate a distal tip 208 of the catheter tubing 202, and the expansile section 204 can be positioned just proximal to the restrictor 206. FIG. 7B provides an end view showing the restrictor 206. The restrictor 206 constricts flow (e.g., fluid embolic agent flow) through the lumen 210, which in turn creates the positive pressure at the expansile section 204 to expand the expansile section 204. In certain implementations, the delivery catheter 200 includes a radiopaque band 214 positioned proximate the distal end of the delivery catheter 200. In some examples, the radiopaque band 214 is positioned along the length 250 of the catheter tubing 202 between the expansile section 204 and the restrictor 206, as shown in FIG. 7A. In this example, the radiopaque band 214 enables a user (such as a surgeon) to view positioning of the very distal end of the expansile section 204 under fluoroscopy. Alternatively, or in addition, the radiopaque band 214 can be positioned distal to the restrictor 206, i.e., closer to distal tip 208. The radiopaque material can include high-density materials such as gold or platinum. Referring again to the expansile section 204, the section can include an expansion-limiting feature, such as a radial fiber 218 built into the wall of the catheter tubing 202 at the expansile section 204. The radial fiber 218 can material with a higher tensile strength than the catheter tubing 202, which would stop the expansile section 204 from expanding radially beyond a predetermined diameter. The radial fiber 218 can comprise a liquid crystal polymer (LCP), polyethylene naphthalate (PEN), ultra-high-molecular-weight polyethylene (UHMWPE), para-aramids, fiberglass, metal wiring (e.g., stainless steel, nitinol, etc.), oriented expanded polytetrafluoroethylene (ePTFE), and the like.

Referring again to the non-expansile section 212, the section can be manufactured such that the section does not expand under a first positive pressure when the expansile section 204 expands under the first positive pressure within the lumen 210. In some implementations, the non-expansile section 212 can be manufactured out of a stiffer elastomeric material than the expansile section 204 such that the non-expansile section 212 resists stretching radially while the expansile section 204 does. The non-expansile section 212 can alternatively or in addition have a thicker section of tubing 202 than the expansile section 204 to resist expansion while the expansile section 204 expands. In some implementations, the non-expansile section 212 can additionally or alternatively include a reinforcing material along the length 250 of catheter tubing 202. This reinforcing material can be placed around the catheter tubing 202 like a sleeve, and/or within the walls of the catheter tubing 202 (e.g., embedded within the material of the catheter tubing 202). In some implementations, and as shown in FIG. 7A, the reinforcing material 220 is positioned along the non-expansile section 212. The reinforcing material 220 can be a braid made from materials such as metal wiring (e.g., stainless steel, nitinol, etc.), para-aramids, fiberglass, carbon fiber, and the like. The reinforcing material 220 can alternatively be a coil made from materials such as metal wiring (e.g., stainless steel, nitinol, etc.), para-aramids, fiberglass, carbon fiber, and the like. It is also contemplated that a second reinforcing material 222 can be positioned on the length 250 of catheter tubing 202 distal to the expansile section 204 to ensure the catheter tubing 202 resists expansion both proximal to and distal to the expansile section 204. The second reinforcing material 222 can be similar to the first reinforcing material 220, although it is also contemplated that the first and second reinforcing materials can comprise different materials.

In certain implementations of the catheter shown in FIGS. 7A-7C, the catheter 200 comprises a liner 602 made from highly oriented PTFE, and an outer polymer jacket 604. The orientation of the polymer chains in the PTFE liner is such that the axial stiffness (or resistance to elongation) of the PTFE liner is considerably greater than that of the outer polymer jacket 604, so that an axial fiber is not required to render the catheter stretch resistant. A wrapped PTFE liner is an example of such a liner material. Specifically, the modulus of elasticity of the preferred liner 602 is more than 1,000 MPa (N/mm2), and preferably it is greater than 1,500 MPa, and most preferably close to or greater than 2,000 MPa. Because this liner 602 can be made with a very thin wall, and is on the inside of the catheter 200, its negative impact on the catheter 200 lateral flexibility can be minimized. The outer catheter jacket(s) 604 have a larger diameter and wall thickness and thus geometrically are configured to have a greater second moment of area. However, because the outer jacket 604 can be made from a polymer with a much lower modulus of elasticity than the inner liner 602, its contribution to both the axial and lateral stiffness of the catheter are lessened. Specifically, the modulus of elasticity of the preferred catheter outer jacket 604 is less than 10% of that of the liner 602, and preferably it is close to or less than 1% of that of the liner 602. This construction is hugely advantageous in that the resultant catheter is extremely flexible, yet also very stretch resistant, so that if the distal tip 208 were to become stuck in the vessel 10 post embolization, it can be safely pulled free without risk of the catheter 200 breaking and portions of it being left behind in the patient.

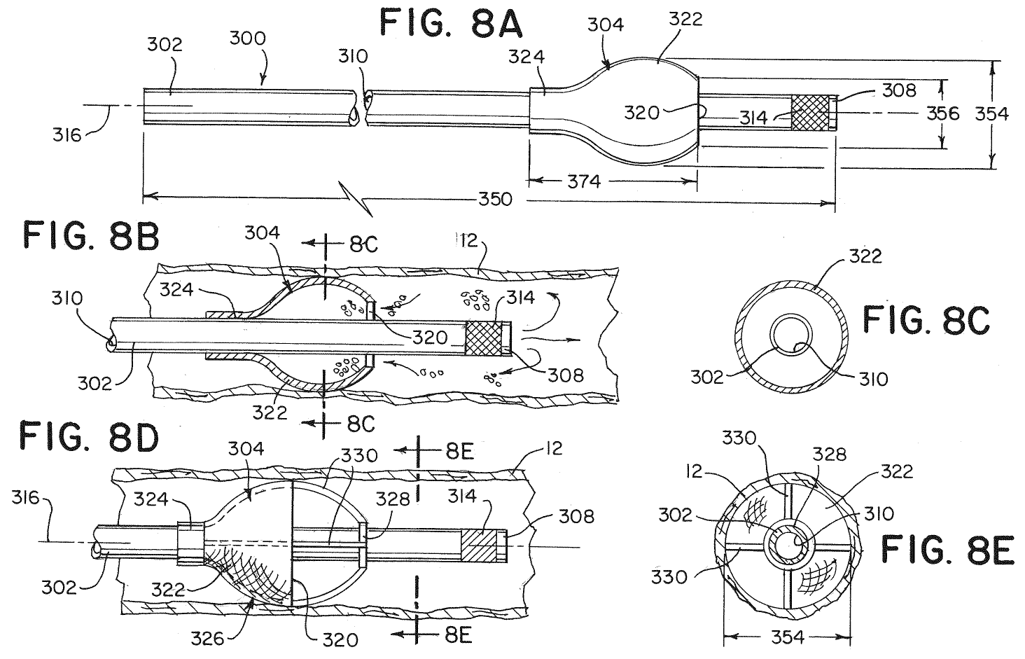

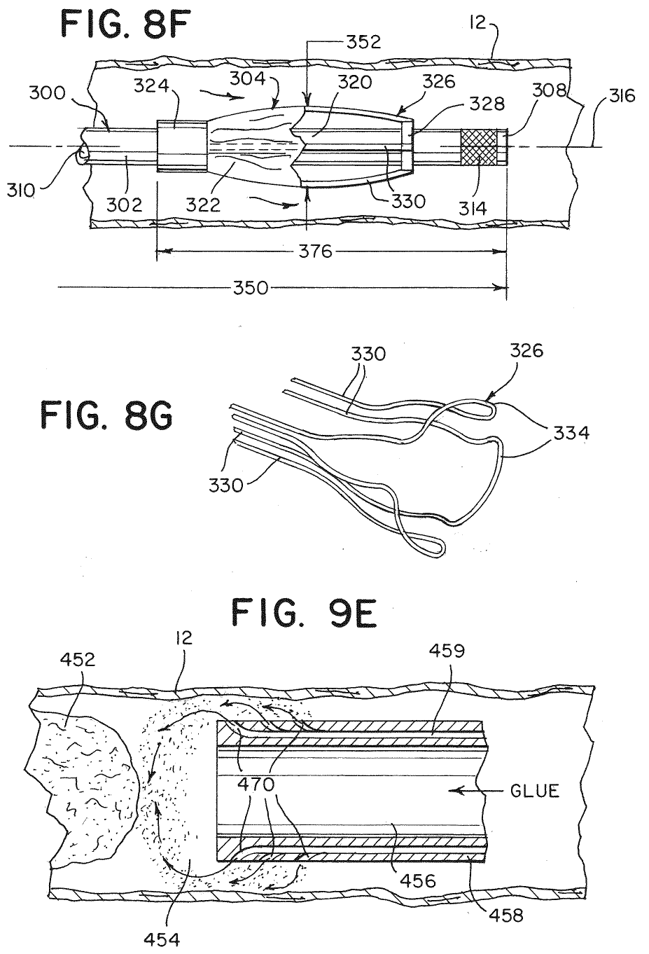

Referring now to FIGS. 8A-8F, the designs therein provide a delivery catheter 300 with an umbrella-style expansile section 304, according to aspects of the present disclosure. The delivery catheters 300 in FIGS. 8A-8F, for example, have a length 350 of catheter tubing 302, and the expansile section 304 is positioned along that length. As was described above, the catheter tubing 302 can include a central lumen 310 disposed therethrough. The expansile section 304 in the examples of FIGS. 8A-8F comprises a fixed proximal collar 324 and a membrane 322 having an open end 320. The expansile section 304 has a collapsed configuration with a first diameter 352 (see FIG. 8F) and an expanded configuration with a second diameter 354 (see FIG. 8D, or the radial cross-sectional view in FIG. 8E). The second diameter 354 is sized to prevent reflux of glue within a vessel 10 proximal to the expansile section 304. As is shown, the fixed proximal collar 324 is attached to the catheter tubing 302, and the open end 320 of the membrane 322 extends distally so that any reflux of the fluid embolic agent from the distal tip 308 is caught within the membrane 322.

FIG. 8A shows an expansile section 304 that has a bulbous shape, wherein the open end 320 of the membrane 322 has an open diameter 356 less than the second diameter 354, the second diameter 354 being closer to the center (lengthwise) of the expansile section 304. As such, the expanded expansile section 304 can contact the vessel wall 102 and the open diameter 356 can be slightly tapered inwardly to aid insertion. The radial force provided by the expanded expansile section 304 can be such that when pressurized from the proximal end (from the left in FIG. 8A), the membrane 322 can collapse. This pressure from the proximal end can be from fluid injections through an intermediate catheter, for example. Delivery catheter 300 can also include a radiopaque band 314 dispositioned proximate the distal tip 308, and the radiopaque band 314 can be similar to the radiopaque band 214 discussed above with reference to FIGS. 7A-7C. FIG. 8B provides a side cross-sectional view of the catheter in FIG. 8A, and FIG. 8C is a radial cross-sectional view of the catheter in FIG. 8B along lines 8C-8C.

Referring again to FIGS. 8D-8F, the expansile section 304 in the example includes a frame 326 extending from the fixed proximal collar 324. The frame 326 includes a sliding distal collar 328 distal to the fixed proximal collar 324. The sliding distal collar 328 is movable to transition the expansile section 304 between the collapsed configuration (see FIG. 8F, wherein the expansile section 304 has a collapsed length 376) and the expanded configuration (see FIG. 8D, wherein the expansile section 304 has an expanded length 374). The membrane 322 is attached to the frame 326 and expands and collapses with the frame 326. The membrane 322 can comprise silicon, polyurethane, polypropylene, polyethersulfone, and/or the like. The material for the membrane 322 is pliable such that the material can be opened as the expansile section 304 opens from the collapsed configuration to the expanded configuration. In some examples, and as shown in FIGS. 8D-8F, the frame 326 can include a plurality of axial bars 330 extending between the fixed proximal collar 324 and the sliding distal collar 328 (e.g., along longitudinal axis 316). The membrane 322 can be affixed to the axial bars 330. To provide the open end 320 of the membrane 322, the membrane 322 need not extend distally entirely to the sliding distal collar 328. The membrane 322 can instead extend from the fixed proximal collar 324 distally to an intermediate point between the fixed proximal collar 324 and the sliding distal collar 328, e.g., at a mid-way point as shown in FIG. 8D.

Referring now to FIG. 8G, the frame 326 can have alternative designs to those shown in FIGS. 8D-8F (FIGS. 8D-8F show bars 330 extending axially along the longitudinal axis 316 and ending at the sliding distal collar 328). The example frame 326 shown in FIG. 8G provides a solution wherein the frame 326 does not include a sliding distal collar 328. In this example, two adjacent axial bars 330 are integrated distally via looped ends 334, thereby providing an atraumatic distal profile for the expansile section 304. The frame 326 can be made from a material capable of recovering its shape automatically once unsheathed into its expanded configuration. The material could be in many forms such as wire, strip, sheet, or tube. In some examples, the frame 326 can include, but is not limited to, Nitinol, stainless steel, MP35N, tungsten, and/or the like or any combination or alloy thereof. In some examples, the material can be made from a memory shape material, such as Nitinol, and the expanded configuration for the expansile section 304 can be made by heat setting the material to the expanded configuration (as shown in FIG. 8G), and then the expansile section 304 can expand when positioned at the treatment site.

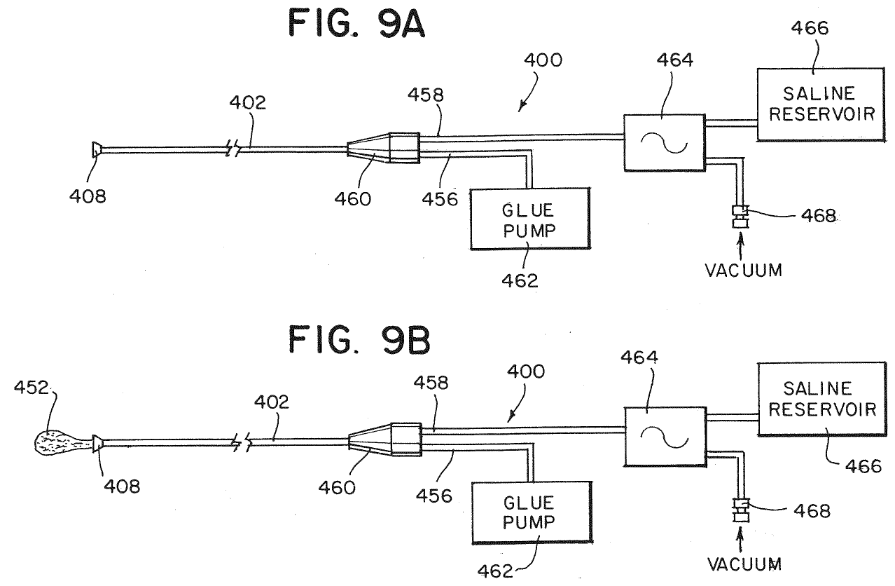

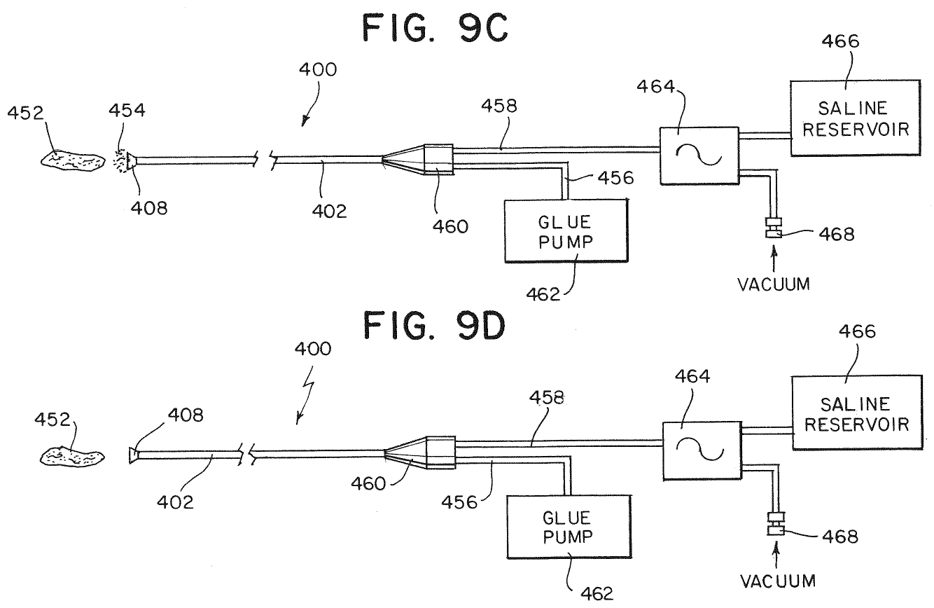

FIGS. 9A-9E are side views of a catheter 400 that utilizes a fluid column 454 to prevent reflux of fluid embolic agent 452, according to aspects of the present disclosure. The example catheter 400 shown in the figures utilizes cyclical pulsing of a second fluid other than glue. The second fluid can include saline, for example, or another fluid such as a dextrose solution (e.g., medical 5% dextrose solution). By cyclically pulsing the second fluid, a punching effect can be used to prevent the fluid embolic agent 452 from flowing proximally. This fluid column 454 can be used as a barrier and then switched off, allowing the catheter 400 to be removed while the set fluid embolic agent 452 remains in place. The catheter 400 includes catheter tubing 402 extending to a distal end 408, wherein the fluid embolic agent 452 and fluid column 454 can exit the catheter 400 proximate the distal end 408. FIGS. 9A-9D show a progression of using the fluid column 454 to prevent reflux. FIG. 9A is a side view of the catheter 400 prior to delivering fluid embolic agent 452 or the second fluid (e.g., saline); FIG. 9B is a side view of the catheter 400 expelling fluid embolic agent 452; FIG. 9C is a side view of the catheter 400 expelling a fluid column 454 to block reflux of the fluid embolic agent 452; and FIG. 9D is a side view of the catheter 400 with the fluid embolic agent 452 distant from the distal end 408, as in this view the fluid embolic agent 452 has been injected at the treatment site and the catheter 400 can be withdrawn.

Catheter 400 can include agent tubing 456 in fluid communication with the distal end 408 of the catheter tubing 402 to provide the fluid embolic agent 452; catheter 400 can include fluid tubing 458 in fluid communication with the distal end 408 of the catheter tubing 402 to provide the fluid for the fluid column 454. Catheter 400 can include an agent pump 462 to drive fluid embolic agent 452 through the agent tubing 456; catheter 400 can include a cyclic pump 464 to drive a second fluid to the distal end 408 of the catheter tubing 402 in cyclic pulses. Referring now to the cyclic pulses, catheter 400 can alternate between driving fluid embolic agent 452 and driving fluid for the fluid column 454. The agent pump 462 and cyclic pump 464 alternate glue and fluid (e.g., saline or another fluid) injection to ensure that the glue continues receive the distal force from the fluid to avoid refluxing. In some implantations, the fluid tubing 458 and the agent tubing 456 can converge at a tubing junction 460 that is in fluid communication with the catheter tubing 402 at its distal end and including with both the fluid tubing 458 and the agent tubing 456 at its proximal end. In certain implementations, catheter 400 can drive the fluid embolic agent 452 and fluid for the fluid column 454 at the same time, thereby producing a punching effect to accompany the delivery of the fluid embolic agent 452.

The agent pump 462 can be connected to a reservoir (e.g., a vial or bag) that includes the fluid embolic agent 452. Alternatively, or in addition, the agent pump 462 can be a positive displacement pump configured to drive the fluid embolic agent 452 continuously through the agent tubing 456 during the cyclic pulses. The cyclic pump 464 can be in fluid communication with a fluid reservoir 466 (e.g., a sterile saline bag). The cyclic pump 464 can also be a syringe comprising the fluid 459 to form the fluid column 454. The syringe design also allows the cyclic pump 464 to provide a degree of vacuum to the treatment area, e.g., the syringe can be depressed inwardly to deliver fluid and pulled out to provide a slight vacuum to prevent an excess amount of fluid 459 (e.g., saline or another fluid) from building proximal to the injected fluid embolic agent 452. It is also contemplated that the cyclic pump 464 can have a vacuum connection 468 to an external vacuum source, which is available in most operating room environments.

Referring now to FIG. 9E, the design shown therein provides an example of how the fluid column 454 does not need to be restricted to only distal to the distal end 408 of the catheter tubing 402. In some examples, the agent tubing 456 can be disposed within the fluid tubing 458, such that fluid 459 for the fluid column 454 flows inside of the fluid tubing 458 and around the agent tubing 456. A distal end of the fluid tubing 458 can include one or more apertures 470 placed near the distal end of the fluid tubing 458 to enable fluid 459 to exit the catheter proximal to the end of the catheter tubing 402. The aperture(s) 470 can be placed 1 mm to 10 mm, e.g., 5 mm, from the end, allowing the fluid column 454 to form along the end of the catheter tubing 402, preventing or limiting reflux of the fluid embolic agent 452 proximally. The apertures 470 can include holes in the catheter tubing 202, which can be rounded holes or any other shape hole to enable egress of fluid through the catheter tubing 202.

Aspects of the designs describe herein can be implemented by any one of the following numbered clauses.
Clause 1: A delivery catheter (100) comprising: a catheter tubing (120) extending a length (500), a proximal portion (220) of the catheter tubing (120) having first diameter (800) and a distal portion (240) of the catheter tubing (120) having a second diameter (810); and a reflux-occluder section (102) having a third diameter (820) greater than the first diameter (800) and the second diameter (810), the reflux-occluder section (102) being disposed along the length (500) of the catheter tubing (120) between the proximal portion (220) and the distal portion (240).
Clause 2: The delivery catheter (100) of Clause 1, wherein the first diameter (800) of the catheter tubing (120) is larger than the second diameter (810) of the catheter tubing (120).
Clause 3: The delivery catheter (100) of Clause 1 or 2, wherein the reflux-occluder section (102) is a bulge in the catheter tubing (120), and the reflux-occluder section (102) is compressible for insertion into a vessel (10) and expandable to the third diameter (820).
Clause 4: The delivery catheter (100) of Clause 3, wherein the catheter tubing (120) comprises a braid (104) extending along the length (500) of the catheter tubing (120).
Clause 5: The delivery catheter (100) of Clause 4, wherein a distal end (112) of the braid (104) is positioned within the proximal portion (220) of the catheter tubing (120).
Clause 6: The delivery catheter (100) of Clause 4, wherein a distal end (112) of the braid (104) is positioned within the reflux-occluder section (102).
Clause 7: The delivery catheter (100) of Clause 4, wherein a distal end (112) of the braid (104) is positioned within the distal portion (240) of the catheter tubing (120).
Clause 8: The delivery catheter (100) of Clause 4, wherein a distal end (112) of the braid (104) comprises a braid attachment (106), the braid attachment (106) being a ring having an atraumatic distal end.
Clause 9: The delivery catheter (100) of Clause 8, wherein the braid attachment (106) comprises a radiopaque material.
Clause 10: The delivery catheter (100) of Clause 1 or 2, wherein the reflux-occluder section (102) is one or more baffles (306/314) extending from the catheter tubing (120).
Clause 11: The delivery catheter (100) of Clause 10, wherein the one or more baffles (306/314) comprise a spiraling baffle (306) that spirals around a longitudinal axis (550) of the delivery catheter (100).
Clause 12: The delivery catheter (100) of Clause 10 or 11, wherein the one or more baffles (306/314) comprise a first spiraling baffle (302) and a second spiraling baffle (304) offset radially from the first spiraling baffle (302).
Clause 13: The delivery catheter (100) of Clause 10, wherein the one or more baffles (306/314) comprise a ring baffle (314) extending radially from the catheter tubing (120).
Clause 14: The delivery catheter (100) of Clause 10 or 13, wherein the one or more baffles (306/314) comprise a first ring baffle (308) having a first baffle diameter (830), and a second ring baffle (310) having a second baffle diameter (840), the first baffle diameter (830) being smaller than the second baffle diameter (840).
Clause 15: The delivery catheter (100) of any of the preceding clauses, wherein the catheter tubing (120) comprises a braid (104) extending along the length (500) of the catheter tubing (120).
Clause 16: The delivery catheter (100) of any of the preceding clauses, wherein the third diameter (820) is less than a diameter of a target a vessel (10).
Clause 17. The delivery catheter (100) of any one of the preceding clauses, wherein the catheter tubing (120) comprises an inner liner (602) and an outer jacket (604), wherein the inner liner (602) comprises highly oriented polytetrafluoroethylene (PTFE), and wherein a modulus of elasticity of the outer jacket (604) is less than 10% of that of the inner liner (602).
Clause 18: A delivery catheter (100) comprising: a catheter tubing (120) extending a length (500) and comprising a braid (104) extending along the length (500) of the catheter tubing (120); and a reflux-occluder section (102) disposed along the length (500) of the catheter tubing (120) between a proximal portion (220) and a distal portion (240) of the catheter tubing (120), wherein a distal end (112) of the braid (104) is positioned within the reflux-occluder section (102).
Clause 19: The delivery catheter (100) of Clause 18, wherein the catheter tubing (120) comprises an axial fiber (402).
Clause 20: The delivery catheter (100) of Clause 18 or 19, wherein: the proximal portion (220) of the catheter tubing (120) comprises a first diameter (800) and the distal portion (240) of the catheter tubing (120) comprises a second diameter (810); and the reflux-occluder section (102) comprises a third diameter (820) greater than the first diameter (800) and the second diameter (810).
Clause 21: A method comprising: advancing a distal tip (110) of a delivery catheter (100) through a main vessel (10) to a position distal to a non-targeted site (14) and proximal to a targeted site (16); positioning a reflux-occluder section (102) proximal to the targeted site (16) such that the reflux-occluder section (102) extends radially from a catheter tubing (120) of the delivery catheter (100) and contacts a vessel wall (12) proximate the targeted site (16); delivering a fluid embolic agent through the catheter tubing (120); limiting, via the reflux-occluder section (102), the fluid embolic agent from refluxing proximal to the reflux-occluder section (102); and withdrawing the delivery catheter (100) from the main vessel (10), wherein the reflux-occluder section (102) comprises an occluder diameter (820) greater than a proximal diameter (800) of a proximal portion (220) of the catheter tubing (120) and also greater than a distal diameter (810) of a distal portion (240) of the catheter tubing (120).
Clause 22: A delivery catheter (200) comprising: a length (250) of catheter tubing (202); a non-expansile section (212) positioned along the length (250); an expansile section (204) disposed along the length (250) distal to the non-expansile section (212); and a restrictor (206) disposed within a lumen (210) of the catheter tubing (202) distal to the expansile section (204), wherein the expansile section (204) has a collapsed configuration with a first diameter (252) and an expanded configuration with a second diameter (254), the second diameter (254) sized to limit reflux of a fluid embolic agent proximal to the expansile section (204) within a vessel (10), and wherein the catheter tubing (202), the non-expansile section (212), and the expansile section (204) share a single lumen (210).
Clause 23: The delivery catheter (200) of Clause 22, wherein the restrictor (206) is a ring of material within the lumen (210) reducing a flow of fluid embolic agent flowing through the lumen (210), and wherein reduction in the flow creates a positive pressure at the expansile section (204) to expand the expansile section (204) from the collapsed configuration to the expanded configuration.
Clause 24: The delivery catheter (200) of Clause 22, wherein the non-expansile section (212) comprises a reinforcing material disposed along the catheter tubing (202) within the non-expansile section (212), the reinforcing material providing radial support to the catheter tubing (202) within the non-expansile section (212) to prevent or reduce expansion of the non-expansile section (212).
Clause 25: The delivery catheter (200) of Clause 24, wherein the reinforcing material (220) is a first braid or a first coil.
Clause 26: The delivery catheter (200) of Clause 25, wherein the reinforcing material (220) is embedded within the catheter tubing (202).
Clause 27: The delivery catheter (200) of Clause 22, further comprising a radiopaque band (214) positioned along the length (250) proximate the restrictor (206).
Clause 28: The delivery catheter (200) of Clause 27, wherein the radiopaque band (214) is positioned along the length (250) between the expansile section (204) and the restrictor (206).
Clause 29: The delivery catheter (200) of Clause 22, wherein the catheter tubing (202) comprises a second reinforcing material (222) positioned around the restrictor (206).
Clause 30: The delivery catheter (200) of Clause 22, wherein the catheter tubing (202) comprises an inner liner (602) and an outer jacket (604), wherein the inner liner (602) comprises highly oriented polytetrafluoroethylene (PTFE), and wherein a modulus of elasticity of the outer jacket (604) is less than 10% of that of the inner liner (602).
Clause 31: A delivery catheter (300) comprising: a length (350) of catheter tubing (302); and an expansile section (304) positioned along the length (350), the expansile section (304) comprising: a fixed proximal collar (324); and a membrane (322) having an open end (320), wherein the expansile section (304) has a collapsed configuration with a first diameter (352) and an expanded configuration with a second diameter (354), the second diameter (354) sized to limit reflux of fluid embolic agent within a vessel (10) proximal to the expansile section (304).
Clause 32: The delivery catheter (300) of Clause 31, wherein the expansile section (304) comprises a frame (326) extending from the fixed proximal collar (324), the frame (326) comprising a sliding distal collar (328), the sliding distal collar (328) movable to transition the expansile section (304) between the collapsed configuration and the expanded configuration.
Clause 33: The delivery catheter (300) of Clause 32, wherein the frame (326) comprises a plurality of axial bars (330) extending between the fixed proximal collar (324) and the sliding distal collar (328), and wherein the membrane (322) is affixed to the axial bars (330).
Clause 34: The delivery catheter (300) of Clause 33, wherein the open end (320) is positioned mid-way between the fixed proximal collar (324) and the sliding distal collar (328).
Clause 35: The delivery catheter (300) of Clause 33, wherein two adjacent axial bars (330) are integrated distally via looped ends (334).
Clause 36: The delivery catheter (300) of Clause 31, wherein the open end (320) has an open diameter (356) less than the second diameter (354).
Clause 37: A delivery catheter (400) comprising: catheter tubing (402) having a distal end (408); agent tubing (456) in fluid communication with the distal end (408) of the catheter tubing (402); an agent pump (462) to drive fluid embolic agent (452) through the agent tubing (456); fluid tubing (458) in fluid communication with the distal end (408) of the catheter tubing (402); and a cyclic pump (464) to drive a second fluid to the distal end (408) of the catheter tubing (402) in cyclic pulses, wherein the cyclic pulses create a fluid column (454) proximate the distal end (408) positioned between the distal end (408) and the fluid embolic agent (452) to limit reflux of the fluid embolic agent (452) proximal to the distal end (408), and wherein the second fluid is a different material than the fluid embolic agent (452).
Clause 38: The delivery catheter (400) of Clause 37 further comprising a tubing junction (460) in communication with the catheter tubing (402) at a first end and (i) the agent tubing (456) and (ii) fluid tubing (458) at a second end.
Clause 39: The delivery catheter (400) of Clause 37, cyclic pump (464) is configured to cycle between driving the second fluid and providing aspiration through the distal end (408) of the catheter tubing (402).
Clause 40: The delivery catheter (400) of Clause 37, wherein the cyclic pump (464) is a syringe comprising the second fluid.
Clause 41: The delivery catheter (400) of Clause 37, wherein the agent pump (462) is a positive displacement pump configured to drive the fluid embolic agent (452) continuously through the agent tubing (456) during the cyclic pulses.

The present disclosure is not necessarily limited to the examples described, which can be varied in construction and detail. The terms “distal” and “proximal” are used throughout the preceding description and are meant to refer to a positions and directions relative to a treating physician. As such, “distal” or distally” refer to a position distant to or a direction away from the physician. Similarly, “proximal” or “proximally” refer to a position near or a direction towards the physician. Furthermore, the singular forms “a,” “an,” and “the” include plural referents unless the context clearly dictates otherwise.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g., “about 90%” may refer to the range of values from 70.1% to 109.9%.

In describing example embodiments, terminology has been resorted to for the sake of clarity. As a result, not all possible combinations have been listed, and such variants are often apparent to those of skill in the art and are intended to be within the scope of the claims which follow. It is intended that each term contemplates its broadest meaning as understood by those skilled in the art and includes all technical equivalents that operate in a similar manner to accomplish a similar purpose without departing from the scope and spirit of the present disclosure. It is also to be understood that the mention of one or more steps of a method does not preclude the presence of additional method steps or intervening method steps between those steps expressly identified. Similarly, some steps of a method can be performed in a different order than those described herein without departing from the scope of the disclosed technology.

What is claimed is:

1. A delivery catheter comprising:
a catheter tubing extending a length, a proximal portion of the catheter tubing having first diameter and a distal portion of the catheter tubing having a second diameter; and
a reflux-occluder section having a third diameter greater than the first diameter and the second diameter, the reflux-occluder section being disposed along the length of the catheter tubing between the proximal portion and the distal portion.

2. The delivery catheter of claim 1, wherein the first diameter of the catheter tubing is larger than the second diameter of the catheter tubing.

3. The delivery catheter of claim 1, wherein the reflux-occluder section is a bulge in the catheter tubing, and the reflux-occluder section is compressible for insertion into a vessel and expandable to the third diameter.

4. The delivery catheter of claim 3, wherein the catheter tubing comprises a braid extending along the length of the catheter tubing; wherein a distal end of the braid is positioned within: (i) the proximal portion of the catheter tubing; (ii) the reflux-occluder section; or (iii) the distal portion of the catheter tubing.

5. The delivery catheter of claim 4, wherein a distal end of the braid comprises a braid attachment, the braid attachment being a ring having an atraumatic distal end.

6. The delivery catheter of claim 1, wherein the reflux-occluder section is one or more baffles extending from the catheter tubing; wherein the one or more baffles comprise: (i) a spiraling baffle that spirals around a longitudinal axis of the delivery catheter; (ii) a first spiraling baffle and a second spiraling baffle offset radially from the first spiraling baffle; (iii) a ring baffle extending radially from the catheter tubing; or (iv) a first ring baffle having a first baffle diameter, and a second ring baffle having a second baffle diameter, the first baffle diameter being smaller than the second baffle diameter.

7. The delivery catheter of claim 1, wherein the catheter tubing comprises an inner liner and an outer jacket, wherein the inner liner comprises highly oriented polytetrafluoroethylene (PTFE), and wherein a modulus of elasticity of the outer jacket is less than 10% of that of the inner liner.

8. The delivery catheter of claim 7, wherein the catheter tubing comprises an axial fiber.

9. A method of using the delivery catheter of claim 1, wherein the method comprises the steps of:
advancing a distal tip of the delivery catheter through a main vessel to a position distal to a non-targeted site and proximal to a targeted site;
positioning the reflux-occluder section proximal to the targeted site such that the reflux-occluder section extends radially from the catheter tubing of the delivery catheter and contacts a vessel wall proximate the targeted site;
delivering a fluid embolic agent through the catheter tubing;
limiting, via the reflux-occluder section, the fluid embolic agent from refluxing proximal to the reflux-occluder section; and
withdrawing the delivery catheter from the main vessel,
wherein the reflux-occluder section comprises an occluder diameter greater than a proximal diameter of a proximal portion of the catheter tubing and also greater than a distal diameter of a distal portion of the catheter tubing.

10. A delivery catheter comprising:
a length of catheter tubing; and
an expansile section positioned along the length, the expansile section comprising:
a fixed proximal collar; and
a membrane having an open end,
wherein the expansile section has a collapsed configuration with a first diameter and an expanded configuration with a second diameter, the second diameter sized to limit reflux of fluid embolic agent within a vessel proximal to the expansile section.

11. The delivery catheter of claim 10, further comprising:
a non-expansile section positioned along the length; and
a restrictor disposed within a lumen of the catheter tubing distal to the expansile section;
wherein the catheter tubing, the non-expansile section, and the expansile section share a single lumen.

12. The delivery catheter of claim 11, wherein the restrictor is a ring of material within the lumen reducing a flow of fluid embolic agent flowing through the lumen, and wherein reduction in the flow creates a positive pressure at the expansile section to expand the expansile section from the collapsed configuration to the expanded configuration.

13. The delivery catheter of claim 11 further comprising a radiopaque band positioned along the length proximate the restrictor.

14. The delivery catheter of claim 10, wherein the expansile section comprises a frame extending from the fixed proximal collar, the frame comprising a sliding distal collar, the sliding distal collar movable to transition the expansile section between the collapsed configuration and the expanded configuration.

15. The delivery catheter of claim 14, wherein the frame comprises a plurality of axial bars extending between the fixed proximal collar and the sliding distal collar, and wherein the membrane is affixed to the axial bars.

16. The delivery catheter of claim 15, wherein the open end is positioned mid-way between the fixed proximal collar and the sliding distal collar.

17. The delivery catheter of claim 15, wherein two adjacent axial bars are integrated distally via looped ends.

18. The delivery catheter of claim 10, wherein the open end has an open diameter less than the second diameter.

19. A delivery catheter comprising:
catheter tubing having a distal end;
agent tubing in fluid communication with the distal end of the catheter tubing;
an agent pump to drive fluid embolic agent through the agent tubing;
fluid tubing in fluid communication with the distal end of the catheter tubing; and
a cyclic pump to drive a second fluid to the distal end of the catheter tubing in cyclic pulses,
wherein the cyclic pulses create a fluid column proximate the distal end positioned between the distal end and the fluid embolic agent to limit reflux of the fluid embolic agent proximal to the distal end, and
wherein the second fluid is a different material than the fluid embolic agent.

20. The delivery catheter of claim 19, wherein the cyclic pump is a syringe comprising the second fluid; and the agent pump is a positive displacement pump configured to continuously deliver the fluid embolic agent during the cyclic pulses.
